(19) United States
(12) Patent Application Publication
(10) Pub. No.: US 2024/0376213 A1
(43) Pub. Date: Nov. 14, 2024

(54) MULTISPECIFIC ANTIBODIES FOR USE IN TREATING DISEASES

(71) Applicant: YEDA RESEARCH AND DEVELOPMENT CO. LTD., Rehovot (IL)

(72) Inventors: Ido AMIT, Rehovot (IL); Rony DAHAN, Rehovot (IL); Yuval SHAPIR, Rehovot (IL); Oren BARBOY, Rehovot (IL)

(21) Appl. No.: 18/692,039

(22) PCT Filed: Sep. 14, 2022
(86) PCT No.: PCT/IL2022/050995

§ 371 Date: Mar. 14, 2024

(30) Foreign Application Priority Data
Sep. 14, 2021 (IL) 286430

(51) Int. Cl. C07K 16/28; A61K 39/00; A61P 35/00; C07K 16/46

(52) U.S. Cl. CPC C07K 16/2851; A61P 35/00; C07K 16/2818; C07K 16/468; A61K 2039/505; C07K 2317/31; C07K 2317/526; C07K 2317/71

(57) ABSTRACT

A multispecific antibody is provided. The antibody comprises a first antigen binding moiety, which specifically binds to an immune checkpoint protein on intratumor T cells and a second antigen binding moiety which specifically binds to a conventional dendritic cell 1 (cDC1). Also provided are uses of such antibodies in cancer treatment.

### RELATED APPLICATIONS

This application claims priority from Israeli Patent Application No. 286430 filed on Sep. 14, 2021 and is hereby incorporated by reference in its entirety.

### SEQUENCE LISTING STATEMENT

The ASCII file, entitled 93949.xml, created on Sep. 14, 2022, comprising 52,075 bytes, submitted concurrently with the filing of this application is incorporated herein by reference.

### FIELD AND BACKGROUND OF THE INVENTION

The present invention, in some embodiments thereof, relates to multispecific antibodies for use in treating diseases.

In recent years, immunotherapy has revolutionized the treatment of numerous types of cancer. A highly promising type of immunotherapy is checkpoint inhibitor therapy. Cancer cells evade the immune system by “hijacking” immune checkpoints, pathways that signal to suppress the activity of this system. Checkpoint inhibitor therapy blocks this inhibitory signaling by immune cells and thereby allows the immune system to attack the malignant cells. Approved checkpoint inhibitors, such as anti-PD1 monoclonal antibodies (mAbs), already constitute a first-line treatment for many cancer types. However, most patients do not respond to these inhibitors, while others rapidly become resistant. Thus, finding ways to increase the efficacy of this therapy is a major research goal.

Tumor cells are destroyed by a subtype of T cells that are cytotoxic. These cells recognize the cancer cells by identifying and binding to class I MHC molecules, an interaction that requires the presence of a glycoprotein termed CD8. Thus, cytotoxic T cells are also known as CD8+ T cells. However, a recent study that explored anti-PD-1 pharmacodynamics within the tumor microenvironment (TME) showed that interactions between T cells and dendritic cells (DCs) are essential for successful anti-PD-1 therapy. Additionally, it showed that conventional type 1 dendritic cells (cDC1s) are crucial for anti-tumor immunity [1]. This T cell-DC crosstalk involves the release of IFN-γ by anti-PD-1-activated T cells and interleukin 12 (IL-12) by tumor-infiltrating DCs, which acts in trans between these cell types to effectively stimulate anti-tumor T-cell immunity.

Background art includes US Patent Application No. 20210130438.

### SUMMARY OF THE INVENTION

According to an aspect of some embodiments of the present invention there is provided a multispecific antibody comprising a first antigen binding moiety, which specifically binds to an immune checkpoint protein on intratumor T cells and a second antigen binding moiety which specifically binds to a conventional dendritic cell 1 (cDC1).

According to an aspect of some embodiments of the present invention there is provided a pharmaceutical composition comprising the multispecific antibody described herein.

According to an aspect of some embodiments of the present invention there is provided a nucleic acid sequence encoding a heavy and/or light chain of the multispecific antibody described herein.

According to an aspect of some embodiments of the present invention there is provided an expression vector comprising the nucleic acid described herein.

According to an aspect of some embodiments of the present invention there is provided a cell transformed with the expression vector described herein.

According to an aspect of some embodiments of the present invention there is provided a method of preparing a multispecific antibody comprising:
(a) culturing the cell described herein under conditions which allow the expression of the multispecific antibody; and
(b) isolating the multispecific antibody from the cell.

According to an aspect of some embodiments of the present invention there is provided a method of treating cancer in a subject in need thereof, the method comprising administering to the subject a therapeutically effective amount of the pharmaceutical composition of claim 20, thereby treating cancer in the subject.

According to some embodiments of the invention, the immune checkpoint protein is selected from the group consisting of PD-1, CTLA-4, TIGIT, LAG-3, TIM-3, ICOS, BTLA, 4-1BB, GITR and OX-40.

According to some embodiments of the invention, the first antigen binding moiety prevents the binding between PD-1 of T cells and PDL-1 expressing cells.

According to some embodiments of the invention, the second antigen binding moiety binds XCR1 or Clec9a.

According to some embodiments of the invention, the immune checkpoint protein is PD-1.

According to some embodiments of the invention, the first antigen binding moiety comprises complementary determining regions as set forth in SEQ ID NOs: 1-3 in a heavy chain with an N to C orientation and complementary determining regions as set forth in SEQ ID NOs: 4-6 in a light chain with an N to C orientation.

According to some embodiments of the invention, the first antigen binding moiety comprises complementary determining regions as set forth in SEQ ID NOs: 11-13 in a heavy chain with an N to C orientation and complementary determining regions as set forth in SEQ ID NOs: 14-16 in a light chain with an N to C orientation.

According to some embodiments of the invention, the second antigen binding moiety comprises complementary determining regions as set forth in SEQ ID NOs: 21-23 in a heavy chain with an N to C orientation and complementary determining regions as set forth in SEQ ID NOs: 24-26 in a light chain with an N to C orientation.

According to some embodiments of the invention, the second antigen binding moiety comprises complementary determining regions as set forth in SEQ ID NOs: 31-33 in a heavy chain with an N to C orientation and complementary determining regions as set forth in SEQ ID NOs: 34-36 in a light chain with an N to C orientation.

According to some embodiments of the invention, an Fc region of said multispecific antibody comprises a mutation that serves to reduce binding of said antibody to FcγRs.

According to some embodiments of the invention, the multispecific antibody comprises knobs-into-holes mutations.

According to some embodiments of the invention, the mutations are in a CH3 domain of a first antibody of said multispecific antibody comprising Y349C/T366S/L368A/Y407V and in a CH3 domain of a second antibody of said multispecific antibody comprising S354C/T366W.

According to some embodiments of the invention, the first moiety comprises amino acid sequences as set forth in SEQ ID NOs: 7 and 8; or SEQ ID NOs: 17 and 18.

According to some embodiments of the invention, the first moiety comprises amino acid sequences as set forth in SEQ ID NOs: 27 and 28; or SEQ ID NOs: 37 and 38.

According to some embodiments of the invention, the multispecific antibody is a bispecific antibody.

According to some embodiments of the invention, the multispecific antibody is for use in treating cancer.

According to some embodiments of the invention, the cancer is characterized by having a T cell: dendritic cell ratio above a predetermined level.

According to some embodiments of the invention, the cancer is selected from the group consisting of: bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D show that effective PD-1 immunotherapy relies on T/DC crosstalk. (A) Comprehensive scRNA atlas of CD8+ T cells following anti-PD-1 treatment from tumor, spleen, and draining lymph node. (B) Subset of cytotoxic T cells that dramatically respond to anti-PD-1 treatment. (C) The DC chemotactic ligand XCL1 is highly and uniquely expressed by the PD-1 responsive subset of CD8+ cytotoxic T cells. (D) Temporary depletion of cDC1 at the active phase of αPD-1 administration using the XCR1-iDTR model abrogates the expected anti-tumor response in the MC38 tumor model. Tumor cells were inoculated subcutaneously. Treatment started 7 days after inoculation, when tumors reached ˜50 mm3. Treatment was injected three times, three days apart. Diphtheria Toxin was injected at 20 ng/g body weight, starting on day 4 after inoculation, every other day. (n=20 mice/group, p=0.0204, p=0.0001, p=0.0001 and p=<0.0001, Student's t-test).

FIGS. 2A-C show bispecific antibody generation and characterization. (A) Illustration of the design of a novel family of bispecific T-cell/DC engagers. Anti-PD-1 is coupled with antibodies targeting specific DC markers. (B) SDS-PAGE analysis of the different constructs. The homogenous band of the non-reduced sample confirms the assembly into a single heterodimer. Reduction of the sample confirms that the bispecific heterodimer comprises the four antibody chains, two from each partner antibody. (C) Analytical size-exclusion chromatograms of bispecific antibodies; numbers indicate native-IgG structure formation molecular weights of the antibodies.

FIGS. 3A-D show single-antigen binding and blocking properties of the bispecific engagers. (A) ELISA demonstrating binding to mPD-1 and mCLEC9A, respectively. Standard binding ELISA titration assay of monospecific and bispecific antibodies to recombinant protein. (B) FACS demonstrating binding to HEK293 cells overexpressing mXCR1. Standard binding titration assay of monospecific and bispecific antibodies. (C) FACS blocking assay using HEK293 cells overexpressing PD-1. Cells were incubated with BiSE or parental mPD-1 mAb, and then with mPD-L1 biotin. PD-1/PD-L1 interactions were detected with conjugated streptavidin. (D) Dose-dependent blocking capabilities of BiSE, control PD-1/Synagis, and parental PD-1 mAb.

FIGS. 4A-B show dual antigen binding properties of the bispecific engagers in vitro. (A) Doublet engagement assay to HEK293 cells expressing each of the target proteins. Standard binding titration assay of the BiSE to a 1:1 mix of overexpressed cells, stained in either CFSE or CellTrace. The percentage of CFSE/CellTrace pairs was quantified from live doublets, and was shown to be dose-dependent. (B) In-vitro assay to PD-1+ T cells and splenocytes. CD8+ cells were isolated from spleens of OT-1 mice, and upregulated PD-1+ after prolonged exposure to OVA in-vitro. Cells were then incubated with naïve splenocytes at a 1:10 ratio with standard titration of PD-1/CLEC9A BiSE or PD-1/Synagis. Doublets were quantified via FACS.

FIGS. 5A-C show dual antigen binding properties of the bispecific engagers in vivo. Evaluation of T/DC doublet formation in the tumor dLN following BiSE treatment in a B16 tumor model. Tumor-bearing mice were treated with PD-1/CLEC9A BiSE, PD-1/Synagis, PD-1 mAb or PBS and dLNs were taken to FACS analysis 24 h later. (A) Doublet formation (n=5, p=0.0012 and p=<0.0001, one-way ANOVA) and (B) cDC1 infiltration (n=5, p=0.0056, p=0.0058, p=0.0008, p=0.0007, one-way ANOVA) were significantly increased in BiSE-treated mice but not in mice treated with the traditional PD-1 mAb. (C) dLNs were taken to ImageStream 24 h following BiSE administration to evaluate doublet formation and BiSE localization. The percent of BiSE+ doublets was calculated from total CD3+ cells (n=2-3 mice, p=0.0018, p=0.001, p=<0.0001, one-way ANOVA). BiSE localized in the T/DC immune synapse as shown in representative images.

FIGS. 6A-C show the activity of the bispecific antibody in vivo. (A) Therapeutic anti-tumor activity of BiSE using a B16 melanoma tumor model, compared to traditional PD-1 blockade in a bispecific format. Tumor cells were inoculated subcutaneously and tumor volume and overall survival were evaluated following treatment. At day 10 of treatment, tumor volume was significantly reduced in PD-1/CLEC9A-treated mice relative to controls (n=10 mice/group, p=0.01, (one-way ANOVA). Overall survival significantly increased in treated mice (p=0.0134, log-rank (Mantle-Cox) test). (B) Anti-tumor activity of BiSE also in a MC38 colon adenocarcinoma model, compared to traditional PD-1 blockade in bispecific format. Treatment with PD-1/XCR1 and PD-1/CLEC9A BiSE resulted in reduced tumor volume at day 6 of treatment relative to controls (n=10 mice/group, p=0.007 and p=0.0003, p=0.0279 and p=0.0042, one-way ANOVA). (C) Evaluation of BiSE activity in a KP lung cancer model. KP cells were injected IV to the tail vein. Antibody injections were on days 15, 21, 24 and 27. Right lung lobes were taken on day 28 to paraffin blocks and H&E slides were prepared. Lung foci were quantified using panoramic viewer software. 3 out of 4 PD-1/CLEC9A BiSE-treated mice were tumor-free.

FIGS. 7A-C show T-cell compartments following BiSE treatment. T-cell compartmentalization and activation in the tumor microenvironment following BiSE treatment using a MC38 adenocarcinoma or B16F10 melanoma tumor model. Mice were treated with the indicated mono- or bi-specific antibody. Treatments were three days apart, and tumors were subjected to FACS analysis 5 days after the third treatment (n=5 mice/group). (A) In the MC38 tumor model, the percentages of CD8+ T-cells (left, p=0.02), T regulatory cells (middle, p=0.004) and CD4+ FoxP3− cells (right) were calculated out of the total percentage of CD45+ immune cells in the TME. The ratios between CD4+CD8 T-cells and Tregs (right, p=0.0403 and p=0.0026) were calculated from the percentages of CD8, CD4 effector and Treg cells out of CD45+ cells in the TME. (B) The percentages of CD8+ T-cells (left, p=0.0167 and p=0.145), T regulatory cells (middle, p=0.0189) and CD4+ FoxP3− cells (right, p=0.0190, p=0.0138) were calculated out of the total percentage of CD45+ immune cells in the TME. The ratios between CD8+, CD4 effectors and Tregs (p=0.0313, p=0.0384) were calculated. PD-1/CLEC9A BiSE showed a significant anti-tumor response when compared to both untreated mice and PD-1 mAb treated mice. (C) The percentages of CD8+ T-cells (left, p=0.0116), T regulatory cells (middle, ns) and CD4+ FoxP3− cells (right, p=0.0163, p=0.0034) were calculated out of the total percentage of CD45+ immune cells in the TME. The ratios between CD8+, CD4 effectors and Tregs (p=0.0027, p=0.0076, p=<0.0001) were calculated. PD-1/CLEC9A BiSE showed a significant anti-tumor response when compared to both untreated mice, PD-1/Synagis and also PD-1/XCR1 treated mice.

FIGS. 8A-D. Doublets and T-cell compartment dynamics in the TME and dLN. T-cell compartmentalization and doublet formation was evaluated in the TME and dLN following BiSE treatment over time using a B16 melanoma tumor model. Mice were treated with PD-1/XCR1, PD-1/CLEC9A, PD-1/Synagis or PBS. BiSE treatments were three days apart, and tumors/dLNs were subjected to FACS analysis 24 hours after each treatment, as well as 5 days after the last treatment (n=5 mice/group). (A) Experimental layout. (B) Doublet formation was enriched in the tumor and dLN following BiSE treatment compared to control groups. (C) The percentages of CD8+ T-cells (left, p=0.008, p=0.012), CD4+ FoxP3− cells (middle, p=0.016, p=0.003) T regulatory cells (right) were evaluated over time. (D) The ratio of T effector/T regulatory ratio was calculated over time from the percentages of CD8+, CD4+ and Tregs out of CD45+ cells in the TME (p=<0.0001, p=0.0027, p=0.0076). PD-1/CLEC9A treated mice showed significant improvement over time when compared to untreated, BiSE control mice and PD-1/XCR1 treated mice.

### DESCRIPTION OF SPECIFIC EMBODIMENTS OF THE INVENTION

The present invention, in some embodiments thereof, relates to multispecific antibodies for use in treating diseases.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

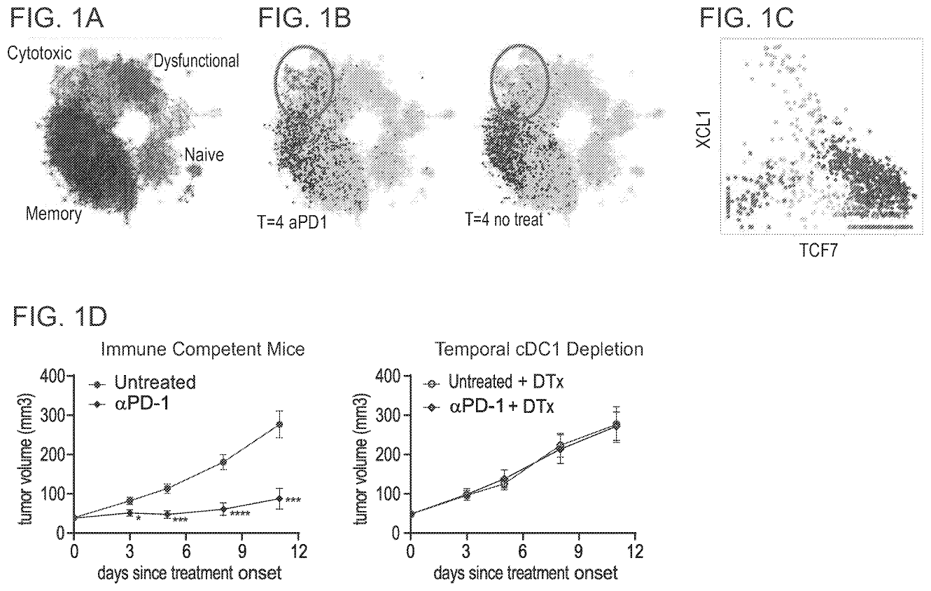

While immunotherapy has already revolutionized the treatment of cancer, success rates of PD-1 based checkpoint inhibition therapy are still limited. Therefore, to obtain molecular and cellular insight into the response of cytotoxic CD8+ T cells to anti-PD-1 treatment, the present inventors analyzed tumor cells using massively parallel single-cell RNA sequencing. Results showed that only a small subset of these tumor-infiltrating cells responds to the treatment. These responding cells expressed high levels of XCL1, which binds to XCR1 expressed on conventional type 1 dendritic cells (cDC1)—FIGS. 1A-C. To further evaluate these findings, the inventors used a XCR1-iDTR mouse model to conditionally deplete cDC1s during the active phase of PD-1 immunotherapy, and found that without cDC1s, the efficacy of anti-PD-1 treatment was completely abrogated (FIG. 1D). The present inventors therefore hypothesized that the low frequency of cDC1 in the tumor microenvironment (TME) is a major limiting factor that compromises effective T cell response to PD-1 inhibition.

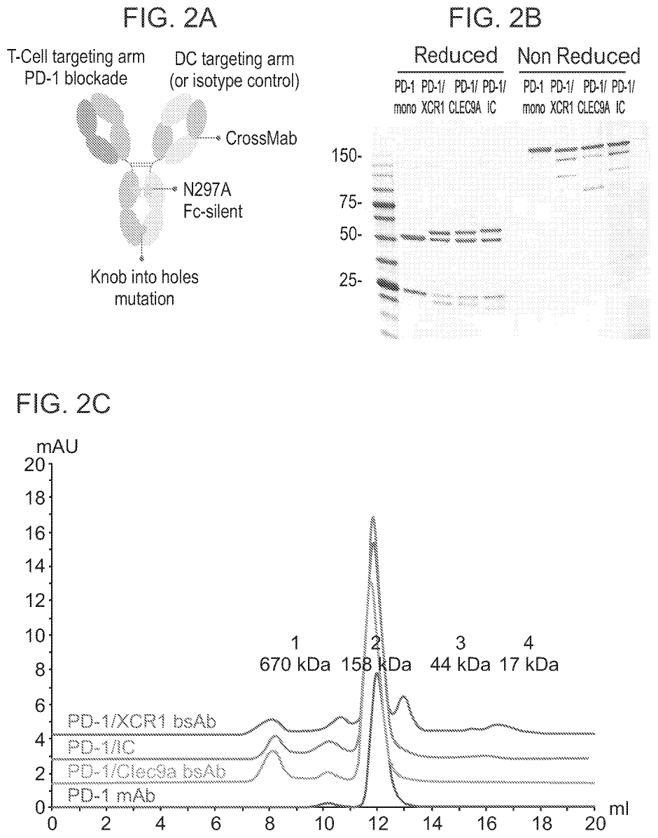

To test this hypothesis and overcome this limitation, the present inventors developed a new approach, referred to herein as “Bispecific Immune Synapse Engagers (BiSE)”. They generated heterodimeric constructs that form a physical connection between PD-1+ T cells and cDC1 by combining a PD-1 blocking monoclonal antibody with cDC1-specific targets (as illustrated in FIG. 2A).

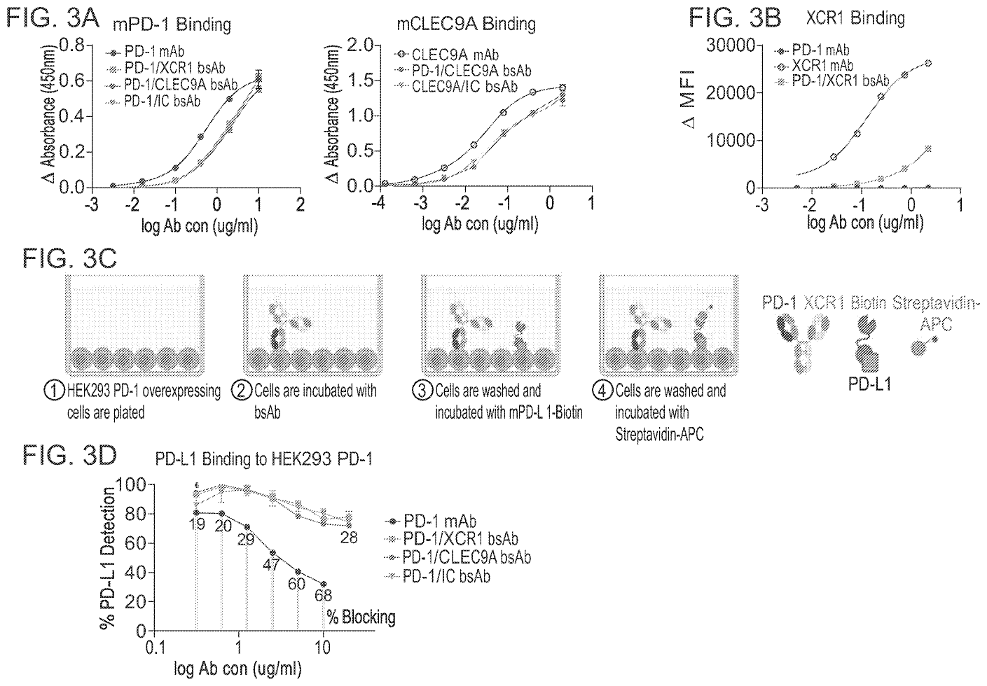

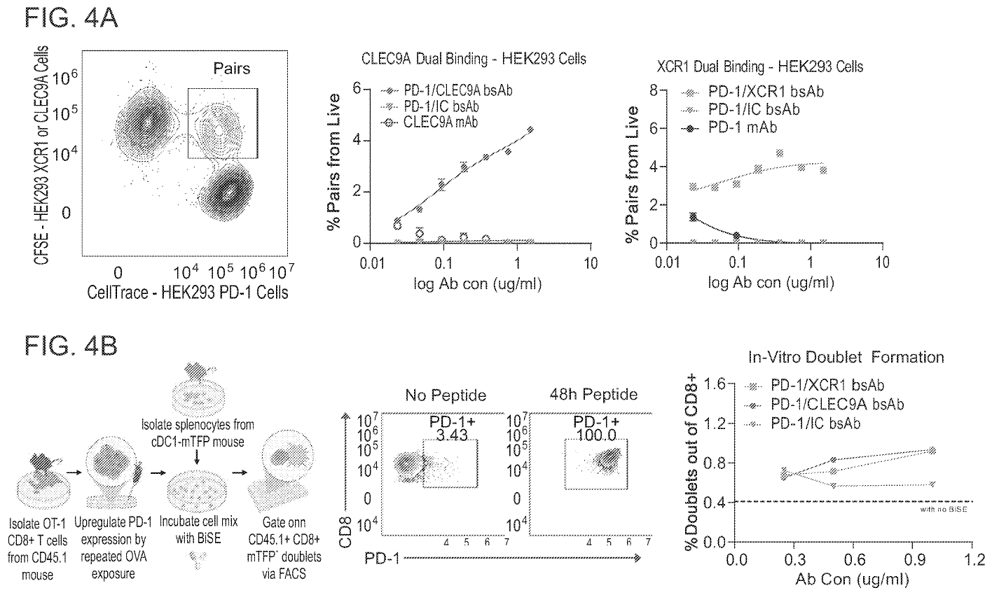

The present inventors show that BiSE reagents bind simultaneously and specifically to their designated targets (FIGS. 3A-B), resulting in T cell-cDC1 doublet formation (FIG. 4A-B), though their PD-1 blocking capability is impaired due to BiSE's monovalent format (FIG. 3C-D).

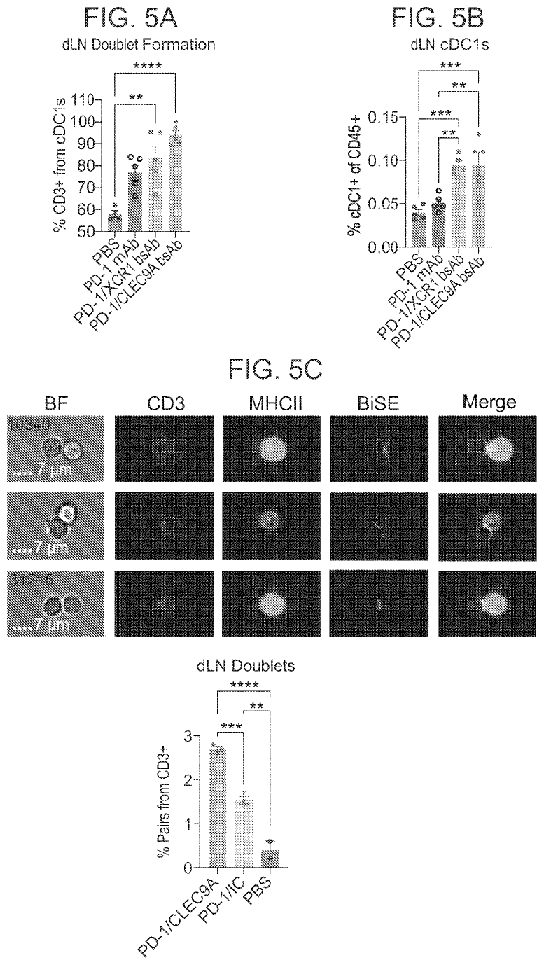

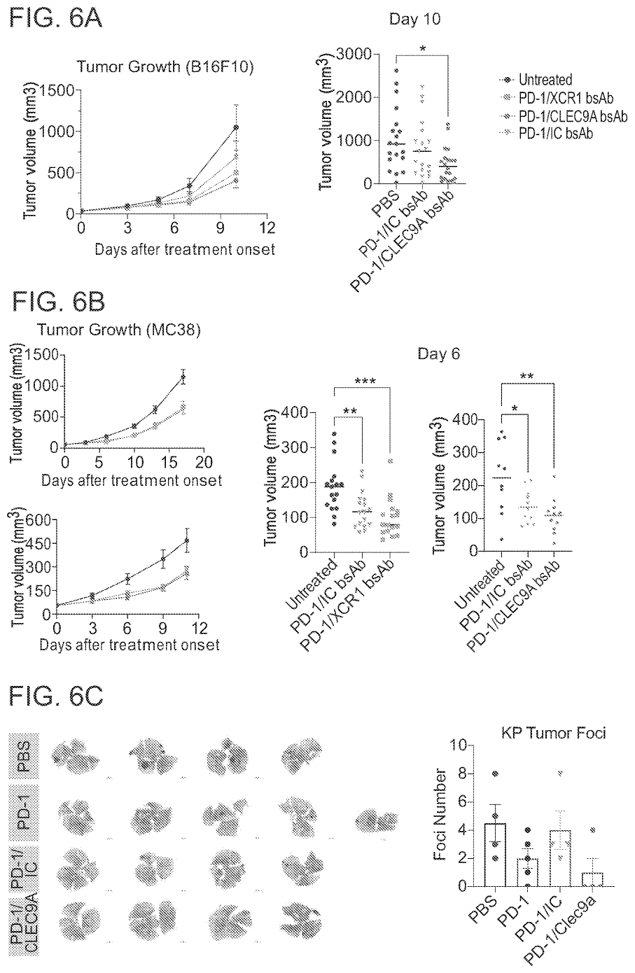

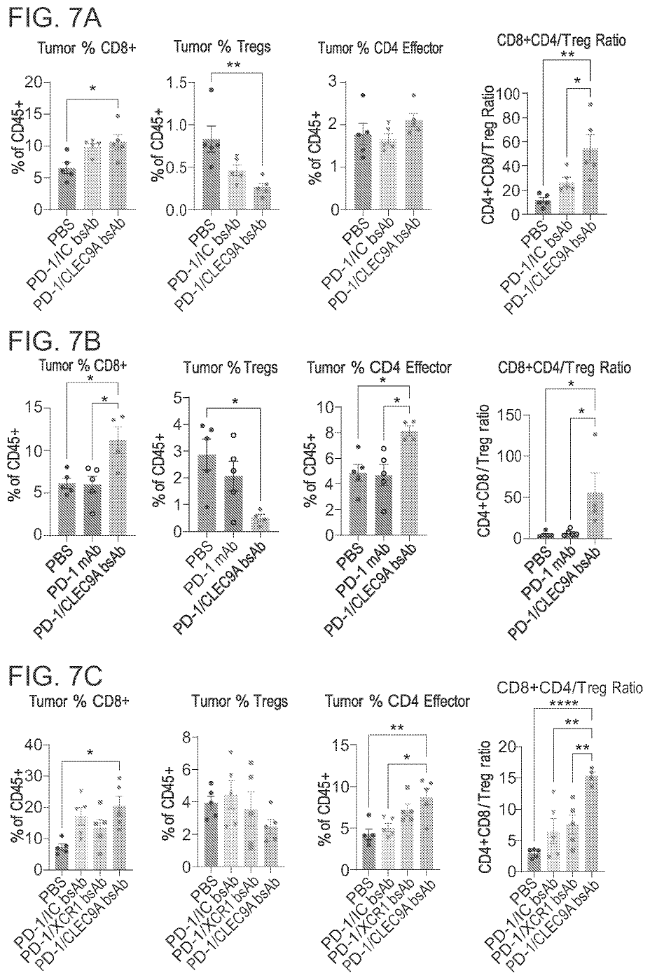

Whilst further reducing the present invention to practice, the present inventors analyzed the anti-tumor activity of this new method in vivo and showed that BiSE-treated tumor model mice exhibit increased doublet formation, mediated by BiSE, and a short-term migration of doublets and cDC1s to the draining lymph nodes (FIG. 5A-C). They also exhibit a significant increase in effector CD8+ and CD4+ T cells at the expense of regulatory T cells in the TME (FIG. 7A-C). These mice displayed increased overall survival and reduced tumor growth compared to untreated mice and to mice treated with isotype control (FIG. 6A-C).

Thus, the present inventors propose BiSE as a new class of immunotherapy, which has the potential to revolutionize the activity of cancer immunotherapy by serving as bispecific T cell—cDC1 synapse engagers.

According to an aspect of the invention there is provided a multispecific antibody comprising a first antigen binding moiety, which specifically binds to an immune checkpoint protein on intratumor T cells and a second antigen binding moiety which specifically binds to a conventional dendritic cell 1 (cDC1).

As used herein, “immune checkpoint” refers to co-stimulatory and inhibitory signals that regulate the amplitude and quality of T-cell receptor recognition of an antigen. In certain embodiments, the immune checkpoint is an inhibitory signal. In some embodiments, the inhibitory signal is the interaction between Programmed Death-1 (PD-1) and Programmed Death Ligand-1 (PD-L1).

As used herein the term “immune checkpoint protein” refers to receptors or their cognate ligands present on the T cell surface that are capable of regulating an immune response to cancer cells. Examples of immune checkpoint proteins include, but are not limited to PD-1, CTLA-4, TIGIT, LAG-3, TIM-3, ICOS, BTLA, 4-1BB, GITR and OX-40.

According to a particular embodiment, the immune checkpoint protein is PD-1 (e.g., human PD-1). The “Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. AAC51773. In one embodiment, the PD-1 has the sequence as specified in GenBank Accession No. AAC51773.

It will be appreciated that antibodies to immune checkpoint proteins may bind human checkpoint proteins and/or mouse checkpoint proteins. Antibodies that bind both human and mouse are typically referred to as “pan-specific antibodies”.

First moiety complementary determining sequences (CDRs), which can be used in the multispecific antibody, according to some embodiments of the invention can be found in the antibodies listed herein below:
Anti PD-1 CDR sequences from clone RMP1-14 are set forth in SEQ ID NOs: 1-6;
Anti PD-1 sequences from clone J43 are set forth in SEQ ID NOs: 11-16;
Pembrolizumab (also named lambrolizumab (MK-3475 or SCH 900475), a humanized monoclonal IgG4 antibody against PD-1);
nivolumab (MDX 1106, BMS 936558, ONO 4538), a fully human IgG4 antibody that binds to and blocks the activation of PD-1 by its ligands PD-L1 and PD-L2;
Dostarlimab (Jemperli) is a humanized IgG4 antibody that binds to and blocks the activation of PD-1 by its ligands.
CTLA-4—ipilimumab, tremelimumab;
TIGIT—Tiragolumab, BMS-986207, COM-902, EOS-448;
LAG-3—BMS-986016 (Relatlimab), FIANLIMAB, favezelimab;
TIM-3—BMS-986258, Sym023, INCAGN02390;
ICOS—MEDI-570, BMS-986226, GSK3359609;
BTLA—TAB004;
4-1BB—utomilumab (PF-05082566), Urelumab (BMS-663513), o GITR-TRX005M, REGN6569, MK-4166;
OX-40—MEDI6469, PF-04518600, BMS 986178.

As mentioned, the multispecific antibody comprises a second moiety which specifically binds to a particular dendritic cell (DC) type-conventional dendritic cell 1 (cDC1).

As used herein “a dendritic cell” (DC) or in plural “dendritic cells” (DCs) refers to cells belonging to a group of cells called professional antigen presenting cells (APCs). DCs have a characteristic morphology, with thin sheets (lamellipodia) extending from the dendritic cell body in several directions. Several phenotypic criteria are also typical, but can vary depending on the source of the dendritic cell. These include high levels of MHC molecules (e.g., class I and class II MHC) and costimulatory molecules (e.g., B7-1 and B7-2), and a lack of markers specific for granulocytes, NK cells, B cells, and T cells. Many dendritic cells express certain markers such as listed below. Dendritic cells are able to initiate primary T cell responses in vitro and in vivo. These responses are antigen specific. Dendritic cells direct a strong mixed leukocyte reaction (MLR) compared to peripheral blood leukocytes, splenocytes, B cells and monocytes. Dendritic cells are optionally characterized by the pattern of cytokine expression by the cell (Zhou and Tedder (1995) Blood 3295-3301). According to a specific embodiment, the multispecific antibody binds immature DCs and possibly mediate they maturation and activation.

As used herein “specifically” refers to a binding preference to DCs as compared to other DC types (plasmacytoids (pDCs), and DCs derived from monocytes (mDCs)).

As used herein, the terms “specific binding.” “selective binding,” “selectively binds,” and “specifically binds,” refer to antibody binding to an epitope on a predetermined antigen but not to other antigens. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE®. 2000 surface plasmon resonance instrument using the predetermined antigen, e.g., recombinant DC marker, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to an “immune checkpoint protein” or a cDC1 marker refers to an antibody that binds to the cell bound marker with a K_{D} of 10⁻⁶ M or less, such as approximately less than 10⁻⁷ M, 10⁻⁸ M, 10⁻⁹ M or 10⁻¹⁰ M or even lower.

According to a specific embodiment, a dendritic cell is characterized by a marker expression selected from the group consisting of Clec9a and XCR1.

According to a specific embodiment, the cDC1s are human cDC1s.

Thus, according to an embodiment of the invention there is provided a multispecific antibody comprising a second moiety comprising complementary determining regions as set forth in SEQ ID NOs: 25-27 in a heavy chain with an N to C orientation and complementary determining regions as set forth in SEQ ID NOs: 28-30 in a light chain with an N to C orientation.

According to a specific embodiment, the second moiety binds Clec9a.

Thus, according to an embodiment of the invention there is provided a multispecific antibody comprising a second moiety comprising complementary determining regions as set forth in SEQ ID NOs: 31-33 in a heavy chain with an N to C orientation and complementary determining regions as set forth in SEQ ID NOs: 34-36 in a light chain with an N to C orientation (CDRs of 10B4).

According to a specific embodiment, the second moiety binds XCR1.

Thus, according to an embodiment of the invention there is provided a multispecific antibody comprising a second moiety comprising complementary determining regions as set forth in SEQ ID NOs: 21-23 in a heavy chain with an N to C orientation and complementary determining regions as set forth in SEQ ID NOs: 24-26 in a light chain with an N to C orientation. (CDRs of MARX10).

Antibodies capable of binding Clec9a are well known in the art. 10B4 is and others are described in U.S. Patent Application No. 20130273150A [15] (e.g., 1F6, 397, and 7H11 are described in [4]). Additional anti human Clec9a antibodies, or CDRs thereof are described in U.S. Patents Application Nos. 20210024637 and 20190352406.

Antibodies capable of binding XCR1 are well known in the art. MARX10 is described in EP EP2641915A1 [5].

According to a specific embodiment, the multispecific antibody comprises SEQ ID NOS: 7 and 8 and SEQ ID NOs: 27 and 28.

According to a specific embodiment, the multispecific antibody comprises SEQ ID NOS: 7 and 8 and SEQ ID NOs: 37 and 38.

According to a specific embodiment, the multispecific antibody comprises SEQ ID NOs: 17 and 18 and SEQ ID NOs: 27 and 28.

According to a specific embodiment, the multispecific antibody comprises SEQ ID NOs: 17 and 18 and SEQ ID NOs: 37 and 38.

The term “antibody” as used in this invention includes intact molecules as well as functional fragments thereof (that are capable of binding to an epitope of an antigen).

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

According to a specific embodiment, the antibody fragments include, but are not limited to, single chain, Fab, Fab′ and F(ab′)₂ fragments, Fd, Fcab, Fv, dsFv, scFvs, diabodies, minibodies, nanobodies, Fab expression library or single domain molecules such as VH and VL that are capable of binding to an epitope of the antigen in an HLA restricted manner.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2, or antibody fragments comprising the Fc region of an antibody.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (sec, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 1996) and the “conformational definition” (see, e.g., Makabe et al., Journal of Biological Chemistry, 283:1156-1166, 2008).

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds);
(vii) Single domain antibodies or nanobodies are composed of a single VH or VL domains which exhibit sufficient affinity to the antigen; and
(viii) Fcab, a fragment of an antibody molecule containing the Fc portion of an antibody developed as an antigen-binding domain by introducing antigen-binding ability into the Fc region of the antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Exemplary methods for generating antibodies employ induction of in-vivo production of antibody molecules, screening of immunoglobulin libraries (Orlandi D. R. et al., 1989. Proc. Natl. Acad. Sci. U. S. A. 86:3833-3837; Winter G. et al., 1991. Nature 349:293-299) or generation of monoclonal antibody molecules by continuous cell lines in culture. These include, but are not limited to, the hybridoma technique, the human B-cell hybridoma technique, and the Epstein-Barr virus (EBV)-hybridoma technique (Kohler G. et al., 1975. Nature 256:495-497; Kozbor D. et al., 1985. J. Immunol. Methods 81:31-42; Cote R J. et al., 1983. Proc. Natl. Acad. Sci. U. S. A. 80:2026-2030; Cole S P. et al., 1984. Mol. Cell. Biol. 62:109-120).

In cases where target antigens are too small to elicit an adequate immunogenic response when generating antibodies in-vivo, such antigens (haptens) can be coupled to antigenically neutral carriers such as keyhole limpet hemocyanin (KLH) or serum albumin [e.g., bovine serum albumine (BSA)] carriers (see, for example, U.S. Pat. Nos. 5,189,178 and 5,239,078]. Coupling a hapten to a carrier can be effected using methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as mice, rabbits, and the like. Suitable protocols involve repeated injection of the immunogen in the presence of adjuvants according to a schedule which boosts production of antibodies in the serum. The titers of the immune serum can readily be measured using immunoassay procedures which are well known in the art.

The antisera obtained can be used directly or monoclonal antibodies may be obtained as described hereinabove.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment.

Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73:119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

As described hereinabove, Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2:97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2:106-10 (1991)].

As mentioned, the antibody fragment may comprise a Fc region of an antibody termed “Fcab”. Such antibody fragments typically comprise the CH2-CH3 domains of an antibody. Fcabs are engineering to comprise at least one modification in a structural loop region of the antibody, i.e. in a CH3 region of the heavy chain. Such antibody fragments can be generated, for example, as follows: providing a nucleic acid encoding an antibody comprising at least one structural loop region (e.g. Fc region), modifying at least one nucleotide residue of the at least one structural loop regions, transferring the modified nucleic acid in an expression system, expressing the modified antibody, contacting the expressed modified antibody with an epitope, and determining whether the modified antibody binds to the epitope. See, for example, U.S. Pat. Nos. 9,045,528 and 9,133,274 incorporated herein by reference in their entirety.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368 812-13 (1994); Fishwild et al., Nature Biotechnology 14, 845-51 (1996); Neuberger, Nature Biotechnology 14:826 (1996); and Lonberg and Huszar, Intern. Rev. Immunol. 13, 65-93 (1995).

Unless otherwise indicated, an immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., human IgG1, exist in several allotypes, which differ from each other in at most a few amino acids.

According to a specific embodiment, the antibody is of an IgG1 isotype. Once antibodies are obtained, they may be tested for activity, for example via ELISA, Western blotting, FACS, dot blot and any other method for antibody qualification.

As used herein a “multispecific antibody” is an antibody that can bind simultaneously to at least two targets that are of different structure, two different antigens or two different epitopes one on the intratumor T cells (at an immune checkpoint protein) and at least one another on a cDC1, as mentioned.

Specificity indicates how many antigens or epitopes an antibody is able to bind; i.e., bispecific, trispecific, quatraspecific. According to a specific embodiment, the antibody is a bispecific antibody.

Using these definitions, a natural antibody, e.g., an IgG, is bivalent because it has two binding arms but is monospecific because it binds to one epitope.

A “bispecific antibody” is an antibody that can bind simultaneously to two targets which are of different structure, one on the intratumor T cells (at an immune checkpoint protein) and another on a cDC1.

Valency indicates how many binding arms or sites the antibody has to a single antigen or epitope; i.e., monovalent, bivalent, trivalent or multivalent. The multivalency of the antibody means that it can take advantage of multiple interactions in binding to an antigen, thus increasing the avidity of binding to the antigen.

Multispecific, multivalent antibodies are constructs that have more than one binding site of different specificity. For example, a diabody, where one binding site reacts with one antigen and the other with another antigen.

As used herein, a “moiety” refers to an antibody component of the multispecific (e.g., bispecific) antibody capable of binding the indicated target.

In order to produce the multispecific antibody of some embodiments of the invention, the present moieties may be modified at the Fc region e.g., the CH3 domain (according to kabat) as well known in the art. Such a modification ensures correct assembly of the multispecific antibody via the heavy chains.

Accordingly, the CH3 domain of one heavy chain is altered, so that within the original interface the CH3 domain of one heavy chain that meets the original interface of the CH3 domain of the other heavy chain within the multispecific antibody, an amino acid residue is replaced with an amino acid residue having a larger side chain volume, thereby generating a protuberance within the interface of the CH3 domain of one heavy chain which is positionable in a cavity within the interface of the CH3 domain of the other heavy chain; and the CH3 domain of the other heavy chain is altered, so that within the original interface of the second CH3 domain that meets the original interface of the first CH3 domain within the trivalent, bispecific antibody an amino acid residue is replaced with an amino acid residue having a smaller side chain volume, thereby generating a cavity within the interface of the second CH3 domain within which a protuberance within the interface of the first CH3 domain is positionable (also known as “the knobs-into-holes” approach by Genentech).

According to a specific embodiment, the amino acid residue having a larger side chain volume is selected from the group consisting of arginine (R), phenylalanine (F), tyrosine (Y), tryptophan (W).

According to a specific embodiment, the amino acid residue having a smaller side chain volume is selected from the group consisting of alanine (A), serine(S), threonine (T), valine (V).

According to a specific embodiment, both CH3 domains are further altered by the introduction of cysteine (C) as amino acid in the corresponding positions of each CH3 domain such that a disulfide bridge between both CH3 domains can be formed.

In a specific embodiment, the bispecific comprises a T366W mutation in the CH3 domain of the “knobs chain” and T366S. L368A, Y407V mutations in the CH3 domain of the “hole chain”. An additional interchain disulfide bridge between the CH3 domains can also be used (Merchant, A. M., et al., Nature Biotech 16 (1998) 677-681) e.g. by introducing a Y349C mutation into the CH3 domain of the “knobs chain” and a E356C mutation or a S354C mutation into the CH3 domain of the “hole chain”. Thus in a another preferred embodiment, the bispecific antibody comprises Y349C. T366W mutations in one of the two CH3 domains and E356C, T366S, L368A, Y407V mutations in the other of the two CH3 domains or the bispecific antibody comprises Y349C, T366W mutations in one of the two CH3 domains and S354C. T366S. L368A, Y407V mutations in the other of the two CH3 domains (the additional Y349C mutation in one CH3 domain and the additional E356C or S354C mutation in the other CH3 domain forming a interchain disulfide bridge) (numbering always according to EU index of Kabat). But also other knobs-in-holes technologies as described by EP 1 870 459A1, can be used alternatively or additionally. A specific example for the bispecific antibody are R409D; K370E mutations in the CH3 domain of the “knobs chain” and D399K; E357K mutations in the CH3 domain of the “hole chain” (numbering always according to EU index of Kabat).

In another embodiment the bispecific antibody comprises a T366W mutation in the CH3 domain of the “knobs chain” and T366S, L368A, Y407V mutations in the CH3 domain of the “hole chain” and additionally R409D; K370E mutations in the CH3 domain of the “knobs chain” and D399K; E357K mutations in the CH3 domain of the “hole chain”.

In another embodiment the bispecific antibody comprises Y349C, T366W mutations in one of the two CH3 domains and S354C, T366S, L368A, Y407V mutations in the other of the two CH3 domains or the bispecific antibody comprises Y349C. T366W mutations in one of the two CH3 domains and S354C. T366S, L368A, Y407V mutations in the other of the two CH3 domains and additionally R409D; K370E mutations in the CH3 domain of the “knobs chain” and D399K; E357K mutations in the CH3 domain of the “hole chain”.

According to a specific embodiment, S354C/T366W mutations are introduced for the 1st mAb (e.g., anti PD-1) and Y349C/T366S/L368A/Y407V mutations are introduced for the 2nd mAb (e.g., anti cDC1).

Alternatively or additionally, for correct heavy-light chain pairing, at least one of the moieties can be expressed in the CrossMab format (CH1-CL swapping).

The basis of the CrossMab technology is the crossover of antibody domains within one arm of a bispecific IgG antibody enabling correct chain association, whereas correct heterodimerization of the heavy chains can be achieved by the knob-into-hole technology as described above or charge interactions. This can be achieved by exchange of different domains within a Fab-fragment. Either the Fab domains (in the CrossMab^{Fab} format), or only the variable VH-VL domains (CrossMab^{VH-VL} format) or the constant CH1-CL domains (CrossMab^{CH1-CL} format) within the Fab-fragment can be exchanged for this purpose. Indeed, for the CrossMab^{CH1-CL} format the respective original light chain and the novel VL-CH1 light chain do not result in undesired interactions with the respective original and VH-CL containing heavy chains, and no theoretical side products can be formed. In contrast, in the case of the CrossMab^{fab} format a non-functional monovalent antibody (MoAb) as well as a non-functional Fab-fragment can be formed. These side products can be removed by chromatographic techniques. In the case of the CrossMab^{VH-VL} format an undesired side product with a VL-CH1/VL-CL domain association known from Bence-Jones proteins can occur between the VL-CH1 containing heavy chain and the original unmodified VL-CL light chain. The introduction of repulsive charge pairs based on existing conserved charge pairs in the wildtype antibody framework into the constant CH1 and CL domains of the wildtype non-crossed Fab-fragment can overcome the formation of this Bence-Jones-like side product in the CrossMab^{VH-VL+/-} format. More details on CrossMab Technology can be found in Klein et al. Methods 154, 1 Feb. 2019, Pages 21-31c.

Alternatively, multispecific e.g., bispecific antibodies described herein can be prepared by conjugating the moieties using methods known in the art. For example, each moiety of the multispecific antibody can be generated separately and then conjugated to one another. A variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. (USA) 82:8648). Other methods include those described in Paulus (1985) Behring Ins. Mitt. No. 78, 118-132; Brennan et al. (1985) Science 229:81-83), and Glennie et al. (1987) J. Immunol. 139:2367-2375). Preferred conjugating agents are SATA and sulfo-SMCC, both available from Pierce Chemical Co. (Rockford, Ill.).

Alternatively or additionally, the conjugation of each moiety of the multispecific antibody can be done via sulfhydryl bonding of the C-terminus hinge regions of the two heavy chains. In a specific embodiment, the hinge region is modified to contain an odd number of sulfhydryl residues, preferably one, prior to conjugation.

According to a specific embodiment, the Fc region of the multispecific antibody is modified to reduce binding specificity to FcγRIIb receptor.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIb, or equivalently RcγRIIB) receptor. Various properties of human FcγRs are summarized in US20170253659.

The majority of innate effector cell types co-express one or more activating FcγR and the inhibitory FcγRIIb, whereas natural killer (NK) cells selectively express one activating Fc receptor (FcγRIII in mice and FcγRIIIA in humans) but not the inhibitory FcγRIIb in mice and humans. Human IgG1 binds to most human Fc receptors and is considered equivalent to murine IgG2a with respect to the types of activating Fc receptors that it binds to.

The modified (mutant) Fc region has one or more mutations corresponding to one or more mutations in a human IgG heavy chain (SEQ ID NO: 41) selected from the group consisting of N297A, S267E (“SE”), S267E/L382F (“SELF”), G237D/P238D/P271G/A330R (“V9”), or G237D/P238D/H268D/P271G/A330R (“V11”) (SEQ ID NO:2), or (“V12”).

Another aspect described herein pertains to nucleic acid molecules that encode the antibodies described herein. The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al, ed. (1987) Current Protocols in Molecular Biology, Greene Publishing and Wiley Interscience, New York. A nucleic acid described herein can be, for example, DNA or RNA and may or may not contain intronic sequences. In a certain embodiments, the nucleic acid is a cDNA molecule.

Nucleic acids described herein can be obtained using standard molecular biology techniques. For antibodies expressed by hybridomas {e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Once DNA fragments encoding V_{H} and V_{L} segments are obtained, these DNA fragments can be further manipulated by standard recombinant DNA techniques, for example to convert the variable region genes to full-length antibody chain genes, to Fab fragment genes or to a scFv gene. In these manipulations, a V_{L}- or V_{H}-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CHI, CH2 and/or CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgGl, IgG2, IgG3, IgG4, IgA, IgE, IgM or IgD constant region, for example, an IgGl region. For a Fab fragment heavy chain gene, the VH-encoding DNA can be operatively linked to another DNA molecule encoding only the heavy chain CH1 constant region.

The isolated DNA encoding the V_{L} region can be converted to a full-length light chain gene (as well as a Fab light chain gene) by operatively linking the VL-encoding DNA to another DNA molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

A variety of prokaryotic or eukaryotic cells can be used as host-expression systems to express the antibodies of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the antibodies of some embodiments of the invention. Conditions of expression in culture depend on the expression system used.

Recovery of the antibody from the culture is effected following an appropriate time in the culture. The phrase “recovering the recombinant antibody” refers to collecting the whole fermentation medium containing the antibody and need not imply additional steps of separation or purification. Notwithstanding the above, antibodies of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

The antibodies, antibody compositions and methods described herein have numerous in vitro and in vivo utilities involving, for example, forming a physical bridge between PD-1+ T cells and cDC1. In a preferred embodiment, the antibodies described herein are human or humanized antibodies. For example, multispecific antibodies described herein can be administered to cells in culture, in vitro or ex vivo, or to human subjects, e.g., in vivo, to enhance immunity in a variety of diseases.

Accordingly, provided herein are methods of treating cancer by administering to a subject in need thereof a therapeutically effective amount of the multispecific antibodies described herein.

As used herein, the term “subject” includes mammals, such as human beings at any age which suffer from a disorder, e.g., cancer, chronic viral infection. According to a specific embodiment, this term encompasses individuals who are at risk to develop the disorder.

In a preferred embodiment, the subject is a tumor-bearing subject and an immune response against the tumor is enhanced. A tumor may be a solid tumor or a liquid tumor, e.g., a hematological malignancy. In certain embodiments, a tumor is an immunogenic tumor. In certain embodiments, a tumor is non-immunogenic. In certain embodiments, a tumor is PD-L1 positive. In certain embodiments a tumor is PD-L1 negative. A subject may also be a virus-bearing subject and an immune response against the virus is enhanced.

Further provided are methods for inhibiting growth of tumor cells in a subject comprising administering to the subject the multispecific antibodies described herein such that growth of the tumor is inhibited in the subject. In certain embodiments, multispecific antibodies described herein are given to a subject as an adjunctive therapy. Treatments of subjects having cancer with multispecific antibodies described herein may lead to a long-term durable response relative to the current standard of care; long term survival of at least 1, 2, 3, 4, 5, 10 or more years, recurrence free survival of at least 1, 2, 3, 4, 5, or 10 or more years. In certain embodiments, treatment of a subject having cancer with multispecific antibodies described herein prevents recurrence of cancer or delays recurrence of cancer by, e.g., 1, 2, 3, 4, 5, or 10 or more years. Treatment with bispecific immune synapse engagers of the present invention can be used as a primary or secondary line of treatment.

Provided herein are methods for treating a subject having cancer, comprising administering to the subject the multispecific antibodies described herein, such that the subject is treated, e.g., such that growth of cancerous tumors is inhibited or reduced and/or that the tumors regress. Multispecific antibodies described herein can be used alone to inhibit the growth of cancerous tumors. Alternatively, multispecific antibodies described herein can be used in conjunction with another agent, e.g., other immunogenic agents, standard cancer treatments, or other antibodies, as described below.

Accordingly, provided herein are methods of treating cancer, e.g., by inhibiting growth of tumor cells, in a subject, comprising administering to the subject a therapeutically effective amount of the multispecific antibodies described herein.

Cancers whose growth may be inhibited using the antibodies of the invention include cancers typically responsive to immunotherapy and cancers that are resistant to immunotherapy. Non-limiting examples of cancers for treatment include squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), ovarian cancer, liver cancer, colorectal cancer, endometrial cancer, kidney cancer (e.g., renal cell carcinoma (RCC)), prostate cancer (e.g. hormone refractory prostate adenocarcinoma), thyroid cancer, neuroblastoma, pancreatic cancer, glioblastoma (glioblastoma multiforme), cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland;

acute myeloid lymphoma, as well as any combinations of said cancers. The methods described herein may also be used for treatment of metastatic cancers, refractory cancers (e.g., cancers refractory to previous immunotherapy, e.g., with a blocking CTLA-4 or PD-1 antibody), and recurrent cancers.

According to a specific embodiment, the cancer is selected from the group consisting of: bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.

The multispecific antibodies described herein can be administered as a monotherapy, or as the only immunostimulating therapy, or it can be combined with an immunogenic agent in a cancer vaccine strategy, such as agonistic antibodies and recombinant proteins and ligands, cancerous cells, purified tumor antigens (including recombinant proteins, peptides, and carbohydrate molecules), cells, and cells transfected with genes encoding immune stimulating cytokines (He et al. (2004) J. Immunol. 173:4919-28).

The multispecific antibodies described herein can be used to enhance antigen-specific immune responses by co-administration of the multispecific antibodies described herein with an antigen of interest, e.g., a vaccine. Accordingly, provided herein are methods of enhancing an immune response to an antigen in a subject, comprising administering to the subject: (i) the antigen; and (ii) the multispecific antibodies described herein, such that an immune response to the antigen in the subject is enhanced. The antigen can be, for example, a tumor antigen, a viral antigen, a bacterial antigen or an antigen from a pathogen.

As previously described, the multispecific antibodies described herein can be co-administered with one or other more therapeutic agents, e.g., a cytotoxic agent, a radiotoxic agent. The antibody can be linked to the agent (as an immuno-complex) or can be administered separate from the agent. In the latter case (separate administration), the antibody can be administered before, after or concurrently with the agent or can be co-administered with other known therapies, e.g., an anti-cancer therapy, e.g., radiation. Such therapeutic agents include, among others, anti-neoplastic agents such as doxorubicin (adriamycin), cisplatin bleomycin sulfate, carmustine, chlorambucil, dacarbazine and cyclophosphamide hydroxyurea which, by themselves, are only effective at levels which are toxic or subtoxic to a patient. Cisplatin is intravenously administered as a 100 mg/ml dose once every four weeks and adriamycin is intravenously administered as a 60-75 mg/ml dose once every 21 days. Co-administration of the multispecific antibodies described herein with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells that would render them unreactive with the antibody.

The multispecific antibody (also referred to in plural as “multispecific antibodies”) can be provided to the subject per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the multispecific antibody accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (multispecific antibody) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

According to a specific embodiment, the dosing of the multispecific antibody can be 0.1-100 mg/kg.

According to a specific embodiment, the dosing of the multispecific antibody can be 0.1-100 mg/kg. According to a specific embodiment, the dosing of the multispecific antibody can be 0.1-80 mg/kg. According to a specific embodiment, the dosing of the multispecific antibody can be 0.1-60 mg/kg. According to a specific embodiment, the dosing of the multispecific antibody can be 0.1-50 mg/kg. According to a specific embodiment, the dosing of the multispecific antibody can be 0.1-40 mg/kg. According to a specific embodiment, the dosing of the multispecific antibody can be 0.1-30 mg/kg. According to a specific embodiment, the dosing of the multispecific antibody can be 0.1-20 mg/kg. According to a specific embodiment, the dosing of the multispecific antibody can be 0.1-10 mg/kg. According to a specific embodiment, the dosing of the multispecific antibody can be 1-100 mg/kg. According to a specific embodiment, the dosing of the multispecific antibody can be 10-100 mg/kg. According to a specific embodiment, the dosing of the multispecific antibody can be 20-100 mg/kg. According to a specific embodiment, the dosing of the multispecific antibody can be 30-100 mg/kg. According to a specific embodiment, the dosing of the multispecific antibody can be 40-100 mg/kg. According to a specific embodiment, the dosing of the multispecific antibody can be 50-100 mg/kg. According to a specific embodiment, the dosing of the multispecific antibody can be 60-100 mg/kg. According to a specific embodiment, the dosing of the multispecific antibody can be 70-100 mg/kg.

According to a specific embodiment, the dosing of the multispecific antibody can be 1-20 mg/kg. According to a specific embodiment, the dosing of the multispecific antibody can be 1-15 mg/kg. According to a specific embodiment, the dosing of the multispecific antibody can be 1-10 mg/kg. According to a specific embodiment, the dosing of the multispecific antibody can be 1-5 mg/kg. According to a specific embodiment, the dosing of the multispecific antibody can be 2-20 mg/kg. According to a specific embodiment, the dosing of the multispecific antibody can be 4-20 mg/kg. According to a specific embodiment, the dosing of the multispecific antibody can be 6-20 mg/kg. According to a specific embodiment, the dosing of the multispecific antibody can be 8-20 mg/kg. According to a specific embodiment, the dosing of the multispecific antibody can be 10-20 mg/kg. According to a specific embodiment, the dosing of the multispecific antibody can be 12-20 mg/kg. According to a specific embodiment, the dosing of the multispecific antibody can be 15-20 mg/kg. According to a specific embodiment, the dosing of the multispecific antibody can be 18-20 mg/kg. According to a specific embodiment, the dosing of the multispecific antibody can be 1-5 mg/kg. According to a specific embodiment, the dosing of the multispecific antibody can be 2-10 mg/kg. According to a specific embodiment, the dosing of the multispecific antibody can be 5-10 mg/kg.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

It is expected that during the life of a patent maturing from this application many relevant antibodies against immune checkpoint proteins will be developed and the scope of the term anti checkpoint antibodies is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., cd. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., cd. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice: C57BL/6 mice were purchased from Envigo. Xcr1-iDTR mice were generated by crossing Rosa-lox-stop-lox-iDTR mice [Jung. S. et al. Immunity 17, 211-220 (2002)] and Xcr1-Cre-mTFP [Buch, T. et al. Nat. Methods 2, 419-426 (2005)]. All in vivo experiments were performed a specific-pathogen free facility. Females were used between the ages of 8-10 weeks, randomized and assigned to experimental groups.

Generation and Characterization of BiSE and Parental mAbs: The variable domains of anti-mouse PD-1 antibodies, clones RMP1-14 and J43 were sequenced from each antibody hybridoma. The sequences of anti-mouse CLEC9A antibody, clone 10B4, and anti-mouse XCR1 antibody, clone MARX10, were taken from patents US20130273150A and EP2641915A1, respectively. The variable heavy and light regions of anti-PD-1 (clones RMP1-14 or J43), anti-XCR1 (clone MARX10) and anti-CLEC9A (clone 10B4) were synthesized based on their published sequences (Syntezza). The variable region sequences of the parental Abs were inserted into mammalian expression vectors with mono human IgG1 or human kappa Fc backbones or into bi-specific vectors. For correct heavy-light chain pairing, the parental DC targeting arm was expressed in the CrossMab format (CH1-CL swapping), while for the PD-1 targeting arm, the wild-type domain architecture was maintained. For heavy chain heterodimerization, point mutations were introduced in the CH3 domain: Y349C/T366S/L368A/Y407V of the DC targeting arm; S354C/T366W of the PD-1 targeting arm. For the generation of the Fc-domain variant (N297A) of human IgG1, site-directed mutagenesis using specific primers was performed by PCR (Agilent Technologies) according to the manufacturer's instructions. Mutated plasmid sequences were validated by direct sequencing (Life Science Core Facility, Weizmann Institute of Science). To produce antibodies, antibody heavy and light chain expression vectors were transiently transfected into Expi293 cells (ThermoFisher). The secreted antibodies in the supernatant were purified by protein G Sepharose 4 Fast Flow (GE Healthcare). Purified antibodies were dialyzed in PBS and sterile filtered (0.22 μm). Purity was assessed by SDS-PAGE and Coomassie staining, and was estimated to be >90%. Size exclusion chromatography (SEC) was performed using a Superose 6 Increase 10/300GL column (GE Healthcare) on an Äkta Pure 25 FPLC system.

Binding ELISA: Binding specificity and affinity of mono and bi-specific Abs were determined by ELISA using recombinant mouse PD-1 (Sino Biological) and mouse CLEC9A (R&D Systems). ELISA plates (Nunc) were coated overnight at 4° C. with recombinant extracellular domain of mouse PD-1 or mouse Clec9a (1 μg/mL/well). All sequential steps were performed at room temperature. After being washed, the plates were blocked for 1 hour with 1×PBS with 2% BSA and were subsequently incubated for 1 hour with serially diluted IgGs (1:5 consecutive dilutions in 1×PBS with 2% BSA). For dual binding ELISA assay, plates were incubated for 1 hour with 1 μg/mL biotinylated mouse PD-1 (Sino Biological). After washing, plates were incubated for 1 hour with horseradish peroxidase-conjugated anti-human IgG (Jackson ImmunoRescarch) or with horseradish peroxidase-conjugated Streptavidin (BioLegend). Detection was performed using a one component substrate solution (TMB), and reactions stopped with the addition of 0.18 M sulphuric acid. Absorbance at 450 nm was immediately recorded using a SpectraMax Plus spectrophotometer (Molecular Devices), and background absorbance from negative control samples was subtracted.

Transfection and establishment of stable cell lines expressing mPD-1, mXCR1 and mCLEC9A: HEK293 cells at 70% confluence were transfected in 6-well cell culture plates (Corning) with 3 μg of pcDNA3.1-PD-1, pcDNA3.1-XCR1, or pcDNA-Clec9a expression vector (Genescript) or empty vector (pcDNA 3.1) using Lipofectamine 2000 (Invitrogen, Carlsbad, CA, USA) following the manufacturer's recommended procedure. After transfection, stable cell lines were established after G418 selection (800 μg/ml) for 14 days. Stable cell lines were cultured in DMEM supplemented with 10% FBS, 800 μg/ml G418, 1% Pen Strep (Gibco). Cultures were maintained in a humidified incubator at 37° C. and 5% CO2-.

HEK293 Overexpressed Cell Binding Assay and Dual Binding Assay: Single cell suspensions were prepared from HEK293 overexpressed cells described above. For surface staining, cells were plated in U-shaped 96-well plates (ThermoFisher) at a concentration of 0.2-1×10⁶ cells in 100 μl PBS. Cells were first stained with LIVE/DEAD™ Fixable blue dead cell stain (ThermoFisher) followed by two washes with PBS. Standard binding titration assay of mAbs and BiSE was performed for 30 minutes on ice. Cells were washed twice with FACS buffer, resuspended in 150 μl FACS buffer, and analyzed by flow cytometry. For dual-antigen engagement FACS assay, standard titration of BiSE was done to HEK293 cells over expressing either XCR1 or CLEC9A, and an additional incubation step was performed with PD-L1 biotinylated recombinant protein (Sino Biological). Dual binding was then detected via Streptavidin (Jackson ImmunoResearch). For dual-binding FACS assay, doublet engagement was assessed between HEK293 cells expressing each of the target proteins. Each cell type was stained in CFSE or CellTrace according to the manufacturer's protocol (ThermoFisher). A mix of 1:1 overexpressed cells stained in CFSE or CellTrace were used in a standard binding titration assay of the BiSE for 30 minutes on ice. Cells were washed twice with FACS buffer, resuspended in 150 μl FACS buffer, and analyzed by flow cytometry. The percentage of CFSE/CellTrace pairs was quantified from live doublets.

PD-1/PD-L1 Blocking Assay: Single cell suspensions were prepared from HEK293-mPD-1 overexpressed cells described above, at a concentration of 0.2-1×10⁶ cells in 100 μl PBS. Cells were incubated with a standard titration of BiSE or parental mPD-1 mAb for 30 minutes on ice, washed twice, and then with mPD-L1 biotin (Sino Biological) for an additional hour on ice. Cells were washed twice, and resuspended in conjugated Streptavidin (Jackson ImmunoResearch) for one hour on ice to detect PD-1/PD-:1 interactions. Cells were then washed twice, resuspended in 150 μl FACS buffer, and analyzed by flow cytometry.

In-Vitro Splenocyte Dual Binding Assay: For PD-1 upregulation on CD8+ T cells, spleens were harvested from OT-1 CD45.1 mice, and single cell suspensions were prepared. Spleens were dissected through a 70 μm nylon cell strainer and then erythrocytes were lysed by 5 min incubation with red blood cell lysis buffer (Sigma-Aldrich) and washed with PBS. CD8+ cells were isolated by negative selection using the EasySep™ Mouse CD8+ T Cell Isolation Kit (Stemcell) according to the manufacturer's protocol. Cells were then seeded at a concentration of 5×105 in a 24-well plate and cultured in complete media (RPMI 1640, 25 mM HEPES, 1% L-Glutamine, 10% FBS, 1% Pen Strep, 1% Non-Essential Amino acids, 1% Pyruvate and 0.05 mM Betamercaptoethanol) with IL-15 (5 ng/ml, Peprotech), IL-7 (5 ng/ml, Peprotech), and 10 mg/ml OVA (257-264) peptide (Sigma-Aldrich). After 24 hours, 10 mg/ml OVA was added additionally to each well. Cells were cultured for 48 hours. Unstimulated controls were cultured in complete media with cytokines but without OVA. Cells were evaluated for PD-1 expression by FACS, and after 48 hours, 100% of cells were PD-1+ compared to unstimulated controls. Once PD-1 expression was established, PD-1+ CD8+ T cells were incubated with splenocytes isolated from XCR1-cre-mTFP mice at a 1:10 ratio in U-shaped 96-well plates (ThermoFisher), approximately 5×105 cells/well. Cells were stained with LIVE/DEAD™ Fixable blue dead cell stain (ThermoFisher) followed by two washes with PBS, and then resuspended in 25 μl FACS buffer with mouse TruStain Fc block (BioLegened), and incubated for 15 minutes at room temperature. Cells were then washed twice, and incubated with BiSE or BiSE control 1:2 serial dilutions for 1 hour on ice in the dark. Cells were washed twice with FACS buffer, and surface antigens were stained in FACS buffer for 30 minutes on ice. Cells were washed twice, resuspened in 150 ul FACS buffer, and analyzed by flow cytometry. Cell populations were defined by the following markers (Biolegend): CD8+ T cells: CD45.1+ (A20), CD8+ (536.7). cDC1s: CD45.1−, CD11c+ (N418), MHC II+ (M5/11415.2), and endogenous mTFP from XCR1-cre-mTFP mice. T cell/DC doublets were quantified from the percentage of T cells. BiSE were identified with R-Phycoerythrin-conjugated anti-human IgG (Jackson ImmunoResearch). Tissue Processing for Flow Cytometry, ImageStream and Single-cell RNA Sequencing Analysis: Lymph nodes were dissected through a 70 μm nylon cell strainer and washed with PBS. Tumors were mechanically dissected into small fragments and transferred to GentleMACS™ C tubes (Miltenyi Biotec) with 0.33 mg/mL DNase I (Roche) and 0.27 mg/mL Liberase TL (Roche) for flow cytometry analysis or 0.1 mg/mL DNase I (Roche) and 1 mg/mL Collagenase IV (Worthington) for single-cell RNA sequencing analysis. Tumors were dissociated twice in the GentleMACS™ Octo Dissociator (Miltenyi Biotec) and the cell suspension was then incubated (37° C. 25 rpm, 40 minutes-flow cytometry/37°° C. 25 rpm, 15 minutes-single-cell RNA sequencing). Tumors were then dispersed through a 70 μm nylon cell strainer and washed with PBS.

Flow cytometry: Single cell suspensions were prepared as described above. For surface staining, cells were plated in U-shaped 96-well plates (ThermoFisher) at a concentration of 0.2-1×10⁶ cells in 100 μl PBS. Cells were first stained with LIVE/DEAD™ Fixable blue dead cell stain (ThermoFisher) followed by two washes with PBS, and then resuspended in 25 μl FACS buffer with mouse TruStain Fc block (BioLegend), and incubated for 15 minutes at room temperature. Surface antigens were stained in FACS buffer for 30 minutes on ice. Then the cells were washed twice with FACS buffer, resuspended in 150 μl FACS buffer, and analyzed by flow cytometry. For intracellular FOXP3 staining, an additional staining step was performed using True-Nuclear™ transcription factor buffer Set Kit (BioLegend) and anti-FOXP3 (BioLegend) according to the manufacturer's instructions. All samples were analyzed on CytoFLEX LX (Beckman Coulter) and FlowJo software. Unless otherwise specified, cell populations were defined by the following markers (BioLegend): cDC1s: CD45⁺, MHC II⁺, CD11c⁺, CD19⁻, CD64⁻, F4/80⁻, Ly6C-, SIRPα⁻. cDC2s: CD45⁺, MHC II⁺, CD11c⁺, CD19⁻, CD64⁻, F4/80⁻, Ly6C-, SIRPα⁺. CD8 effectors: CD45⁺, CD11b⁻, CD3⁺, CD8⁺, CD4⁻, CD4 effectors: CD45⁺, CD11b⁻, CD3⁺, CD8⁺, CD4⁺, FOXP3⁻. Tregs: CD45⁺, CD11b⁻, CD3⁺, CD8⁺, CD4⁺, FOXP3⁺. T-cell/cDC1 Doublets were defined by the following markers: CD45⁺, MHC II⁺, CD11c⁺, CD19⁻, CD64⁻, F4/80⁻, Ly6C-, SIRPα⁻, CD3⁺.

ImageStream: WT mice were inoculated with B16F10 tumors as described. On day 11. mice were treated by intraperitoneal injection with 500 ug/mouse of BiSE. Mice were sacrificed on day 12, and tumor-draining lymph nodes were removed. Single cell suspensions were prepared as described above. Cells were first stained with LIVE/DEAD™ Fixable violet dead cell stain (ThermoFisher) followed by two washes with PBS, and then resuspended in 25 μl FACS buffer with mouse TruStain Fc block (BioLegened), and incubated for 15 minutes at room temperature. Surface antigens were stained in FACS buffer for 30 minutes on ice. Then the cells were washed twice with FACS buffer, resuspended in 30 μl FACS buffer, and analyzed by ImageStream. Cell populations were defined using the following markers (BioLegend): DCs: MHC II+, CD11c+. T cells: CD3+. BiSE were identified with R-Phycoerythrin-conjugated anti-human IgG (Jackson ImmunoResearch), or labeled prior to injection with the SAIVI Alexa Fluor 647 Antibody Labeling Kit (ThermoFisher). Cells were imaged using a multispectral Imaging Cytometer (ImageStreamX mark II imaging flow-cytometer; Amnis, Part of EMD Millipore). At least 5×103 cells were collected from each sample and data were analyzed using the manufacturer's image analysis software (IDEAS 6.2; Amnis).

Tumor Challenge and Treatment: Tumor cell lines were maintained in a humidified incubator at 37° C. and 5% CO₂, and cultured in complete RPMI medium containing 25 mM HEPES, 1% L-Glutamine, 10% FBS, 1% Pen Strep, 1% Non-Essential Amino acids, and 1% Pyruvate. MC38 (2×10⁶) and B16-F10 (4×10⁵) were implanted subcutaneously on the right flank of mice, and tumor volumes were blindly measured every 2-3 days with an electronic caliper. Volume is reported using the formula (L₂²*L₁)/2, where L₁ is the longest diameter and L₂ is the shortest diameter. Seven to 10 days after tumor inoculation, when the sum of tumor length and width reached approximately 50 mm³, mice were randomized by tumor size (day 0), and received treatment by intraperitoneal injection as described for each experiment. WT mice were treated with 500 μg BiSE or control PBS at days 0, 3 and 6. Mice were monitored for 8-20 days after treatment initiation, or until the majority of the untreated control group had to be sacrificed due to the Weizmann Institute of Science IACUC limitation for tumor size. For the KP model, mice were injected intravenously with 5×105 KP cells and received treatment by intraperitoneal injection and treated with 200 μg of in-house anti-mouse PD-1 IgG1-N297A mAb (clone RMP1-14), 500 μg BiSE or control PBS at days 15, 18, 21, 24 and 27.

H&E Staining: WT KP-bearing mice were treated by intraperitoneal injection with 500 ug/mouse of BiSE or monospecific abs after tumor inoculation as previously described. After 28 days, lungs from treated animals were harvested and placed in 4% paraformaldehyde (PFA) overnight, and then paraffin processed and stained with hematoxylin and eosin (H&E) at the Histology & Pathology unit of the Weizmann Institute of Science. Slides were scanned using a Pannoramic scan II scanner (3DHISTECH), and images were obtained and analyzed with Case Viewer software.

Cell-depletion studies: XCR1+cDC1s were depleted starting 4 days following tumor inoculation and during the entire treatment period at a dose of 20 ng/g every other day, until 4 days after treatment completion. Depletion efficiency was assessed by either flow cytometry analysis or blood platelet count.

Data and Statistical analysis: Flow cytometry data analysis was performed using FlowJo v.10.6.2; all other data analyses were performed using GraphPad Prism (GraphPad Software). Quantitative data are presented as means±s.e.m. unless otherwise indicated. When geometric mean intensity is presented, MFI is shown; when the value is the florescence intensity of the Ab deduced from an isotype control, delta geometric mean intensity is presented (ΔMFI). Although no statistical method was used to predetermine sample size, mouse numbers were taken into consideration for in vivo studies to ensure that biological effects would be detected, and to enable comparison between groups, and were determined based on results of preliminary experiments. Group allocation was randomized for all in vivo experiments as described above. Tumor measurements were performed blindly; for other in vivo and in vitro studies the researchers were not blinded to treatment group when performing the experiments. For each dataset, normality of the population and/or population residuals (Gaussian distribution) was confirmed using Shapiro-Wilk and/or D'Agostino-Pearson testing. For normal distributions, one-way analysis of variance (ANOVA) with Tukey's post hoc test was used to compare all groups with three or more treatments. When two groups were compared, an unpaired two-tailed Student's t-test (two-tailed, unequal variance) was used to determine statistical significance. When data were not normally distributed, a nonparametric test was used-Kruskal-Wallis with Dunn's post hoc test for multiple comparisons or Mann-Whitney test when two groups were compared. P<0.05 was considered as significant in all statistical tests and is represented in figures as follows: *P<0.05, **P<0.01, ***P<0.001, ****P<0.0001.

### EXAMPLE 1

### A Subset of Tumor-Specific CD8+ T Cells Respond to Anti-PD-1 Treatment

Most cancer patients are unresponsive to anti-PD-1 checkpoint inhibition treatment. To fully characterize the cellular and molecular pathways of CD8+ T cell response after anti-PD-1 treatment, massively parallel single-cell RNA sequencing was conducted on samples from tumor, spleen, and draining lymph nodes (dLNs). The scRNA-seq data revealed that the cytotoxic T cells that responded most robustly to PD-1 blockade within the TME were a unique tumor-specific subset of cells. These cells expressed high levels of the DC chemoattractant XCL1, a specific ligand of the XCR1 receptor expressed exclusively on cDC1, which has been shown to be crucial for anti-tumor immunity. In contrast, dysfunctional T-cells expressed high levels of PD-1 without any XCL1 expression (FIGS. 1A-C). These results suggested that the effectiveness of anti-PD-1 treatment could depend on interaction between CD8+ T cells and cDC1.

To test these results, cDC1s were conditionally depleted using the XCR1-cre-iDTR mouse line during the active phase of anti-PD-1 immunotherapy in an MC38 colon adenocarcinoma tumor model. The significant anti-tumor effect of anti-PD-1 was completely abrogated following cDC1 depletion (FIG. 1D), emphasizing the importance of T/DC crosstalk in the TME during anti-PD-1 immunotherapy.

### EXAMPLE 2

### Generation of T Cell/Dendritic Cells BiSE

Based on the scRNA-seq profiling of the TME post treatment and result from genetic model for inducible depletion of cDC1, it was hypothesized that formation of immune synapses between CD8+ T cells and cDC1 would improve the potency of anti-PD-1 treatment. To test this hypothesis, multiple PD-1/cDC1 BiSE, (i.e. reagents that form physical connections with both PD-1 and cDC1) were generated. For the cDC1 arm, cDC1-restricted surface markers XCR1 or CLEC9A were used together with the variable regions of the MARX-10 clone or the 10B-4 clone, respectively. Both targets are similarly conserved in mice and humans. For the PD-1 targeting arm, the variable region of the well-characterized anti-PD-1 mAb clone RPM1-14 was used. The biochemical properties of the BiSE were investigated as well as their dual specificity to the desired targets. As controls, monomeric immunoglobulin G (IgG) versions of the parental mAbs composing each BiSE were generated, as well as a PD-1/Synagis construct, an isotype control in a bispecific format that targets PD-1 with only one arm of the antibody, without engaging DCs.

To produce the desired bispecific antibody combination, it is necessary to assemble the four heavy and light chains of the existing antibodies correctly. The knob-into-holes technology [2] was used, which identifies point mutations in the CH3 domains of the heavy chains, to enable heterodimerization of the desired heavy chains. CrossMab technology [3], i.e. the exchange of heavy-chain CH1 and light-chain CL1 domains of one of the two antibodies composing the bispecific antibody, to ensure the correct association of the light chains with their cognate heavy chains. The Fc of the BiSE was mutated (N297A) to abrogate FcγR binding. The four constructs of each BiSE were co-transfected into HEK293 cells for production and purified from the cell medium by immobilized protein G affinity columns. The bispecific antibodies were characterized for purity and homogeneity by SDS-PAGE and analytical size-exclusion chromatography (SEC) (FIGS. 2A-C). Results showed that the construct was properly assembled into the desired heterodimer comprising two chains from each antibody, as well as the purity of the obtained constructs.

### EXAMPLE 3

### Examination of the Binding and Blocking Ability of BiSE

Next, the binding ability of each arm was evaluated by either ELISA or FACS, using transfected HEK293 cells expressing either PD-1, XCR1, or CLEC9A and recombinant proteins. Results showed binding to all three targets of any mono-or bi-specific antibody with the relevant specificity (FIG. 3A,B). The ability of BiSE to block the interaction between PD-1 and its ligand PD-L1 was tested using a FACS-based PD-1 blocking assay (FIG. 3C,D). As seen in FIG. 4B, both XCR1- and CLEC9A-BiSE reduced PD-1/PD-L1 interaction.

### EXAMPLE 4

### Examination of the Dual Binding Ability of BiSE

The ability of BiSE to bind both targets simultaneously, enforcing crosstalk between PD-1+ and XCR1+ or CLEC9A+ cells and thereby modeling T-cell/DC interaction, was then assessed by a dual-binding FACS assay (FIG. 4A), quantifying the percentage of CFSE/CellTrace-stained pairs. Results showed effective simultaneous binding to PD-1 and XCR1 and to PD-1 and CLEC9A in a dose-dependent manner. These results indicate that BiSE can bind concurrently in trans (i.e., on two different cells) to PD-1 and either cDC1 surface markers. Dual binding was also assessed on splenocytes in an in-vitro assay. CD8+ T cells were isolated from OT-1 CD45.1 mice and upregulated PD-1 in culture after repeated OVA exposure. They were then incubated with naïve splenocytes with a standard BiSE titration and T/DC doublets were analyzed via FACS. BiSE mediated dose-dependent doublet formation of the splenocytes, showing effective binding to both PD-1 and XCR1/CLEC9A on mice target cells (FIG. 4B).

### EXAMPLE 5

### Examination of In Vivo Dual Binding Activity of BiSE

To test BiSE's ability to form the T/DC immune synapse in vivo, mice were inoculated with B16F10 tumors, in order to induce T cell exhaustion, increase PD-1 expression on CD8+ T cells, and model the TME. Following tumor establishment, the mice were treated with BiSE and T/DC synapse formation was evaluated one day later. BiSE-treated mice showed increase doublet formation in the dLN (FIG. 5A) as well as an increase in the percentage of cDC1s from total immune cells in the dLN, showing that BiSE may induce cDC1 migration to the dLN upon administration (FIG. 5B). To assess if BiSE mediate the increase in immune synapse formation, tumor-bearing mice were treated with BiSE labeled with a fluorophore. One day later, doublet formation was evaluated by ImageStream. BiSE localized within the immune synapse in PD-1/CLEC9A treated mice and increased synapse formation significantly compared to PD-1/Synagis treated mice (FIG. 5C).

### EXAMPLE 6

### Examination of In Vivo Antibody Activity of BiSE

To test the in vivo engagement of the immune synapse by BiSE, the B16F10 tumor mouse model, which is resistant to traditional anti-PD-1 monotherapy, as well as the MC38 colon adenocarcinoma model were used. In the B16F10 tumor model, treatment with the PD-1/CLEC9A BiSE resulted in significant increase in overall survival and reduced tumor growth compared to untreated mice and to mice treated with PD-1/Synagis isotype control (FIG. 6A). In the MC38 tumor model, treatment with PD-1/XCR1 BiSE resulted in a moderate anti-tumor effect at 6 days of treatment (FIG. 6B). In the KP lung tumor model, treatment with PD-1/CLEC9A BiSE resulted in the complete elimination of KP lung tumor foci in 3 out of 4 mice (FIG. 6C).

### EXAMPLE 7

### Examination of In Vivo TME T Cell Modulatory Activity of BiSE

Lastly, T-cell compartmentalization and activation in the TME and dLN following BiSE treatment was evaluated using an MC38 colon adenocarcinoma tumor model and a B16F10 melanoma model. Mice were analyzed 5 days following the third BiSE treatment. Treatment with BiSE resulted in a significant increase in cytotoxic CD8+ and effector CD4+ T cells, a significant reduction in T regulatory cells (Tregs) and a significantly increased CD8/Treg ratio, as compared to untreated mice (FIG. 7A,B,C). CD4 effector/Treg ratio and CD8/CD4 effector/Treg ratio were also significantly increased following BiSE treatment compared to the PD-1/Synagis-treated control mice (FIG. 7A.C), and also PD-1 mAb-treated mice (FIG. 7B) showing the added effect of immune synapse formation in the TME

### EXAMPLE 8

### Examination of the BiSE Timeline

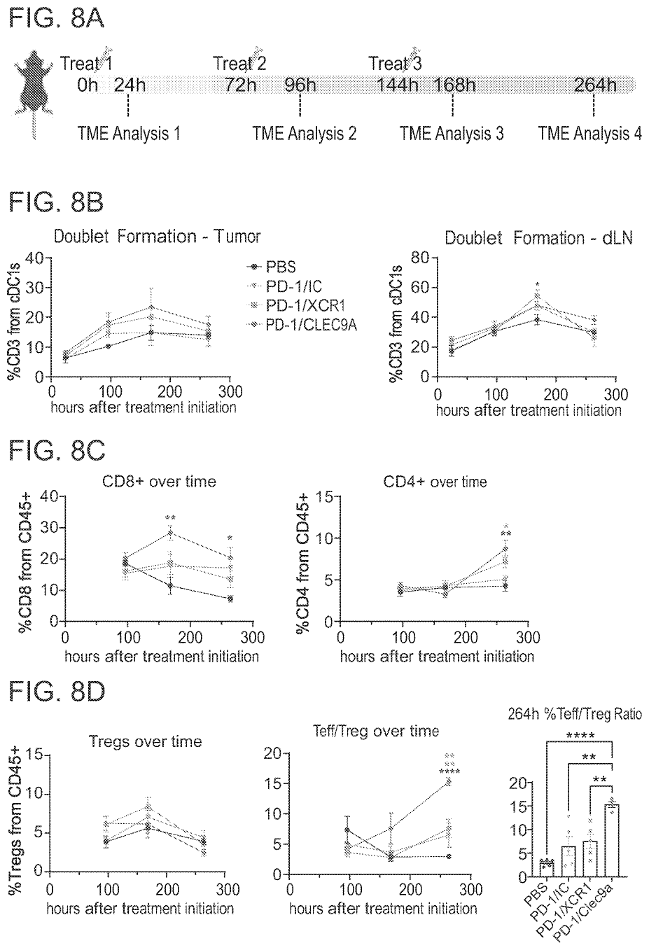

Finally, doublet formation in the TME and dLN as well as T-cell compartmentalization and activation in the TME was evaluated following BiSE treatment and scaling over four timepoints, using a B16 tumor model. Mice were harvested 24 hours after the first, second and third BiSE injection, as well as 5 days after the third BiSE injection. Treatment with BiSE resulted in improved T-cell/cDC1 doublet formation in the tumor and dLN, as well as a significant increase in cytotoxic CD8+ T cells, CD4+ cells, and a significantly increased T effector/Treg ratio compared to both untreated and isotype control groups, once more emphasizing the added benefit of immune synapse formation in the TME (FIG. 8A-D).

### EXAMPLE 9

### Qualifying Antibodies which Bind Human Cells

Dual Binding/PD-1 blocking assays—use HEK293 cells transfected with human antigen targets.

PD-1/PD-L1 Blockade Bioassay—Use a premade kit to assess PD-1 blocking and MOA using BiSE or control Ab.

### Doublets, DC/T Activation and Cytokine Secretion Co-Culture Assays

PBMCs from human blood donors are isolated. T cells and DCs are enriched using dedicated antibodies against CD8 and CD14, respectively immature DCs are differentiated by using reagents such as GM-CSF and IL-4 and incubated with BiSE or control antibody. Effect is determined by IFNγ/IL-12 cytokine release via cytokine ELISA, DC/T activation markers via FACS, doublet formation.

### In-Vitro Imaging of DC:T Interaction

Seed enriched DCs (from human PBMCs) on coated coverslips and co-culture with prestimulated T cells in the presence of BiSE or control antibodies. Analyze by immunofluorescence.

### T Cell Proliferation Assay

Enrichment of PBMCs for T cells and labeling with CFSE/CellTrace. Stimulation with anti-CD3 antibody and culturing in the presence of DCs and BiSE/control antibody for a number of days. Analysis of CFSE/CT dilution via FACS. Harvesting of supernatants for cytokine analysis 48 h after the start of culture.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### (Other References are Cited Throughout the Application)

1. Garris C S, Arlauckas S P, Kohler R H, et al. Immunity. 2018;49(6):1148-1161.e7. doi:10.1016/j.immuni.2018.09.024
2. Merchant, A. M. et al. Nat. Biotechnol. 16, 677-681 (1998).
3. Schaefer W, Regula J T, Bähner M, et al. Proc Natl Acad Sci U S A. 2011;108(27):11187-11192. doi:10.1073/pnas.1019002108
4. D. Sancho et al., “Tumor therapy in mice via antigen targeting to a novel, DC-restricted C-type lectin,” J. Clin. Invest., vol. 118, no. 6, pp. 2098-2110, 2008.
5. A. Bachem et al., “Expression of XCR1 characterizes the Batf3-dependent lineage of dendritic cells capable of antigen cross-presentation,” Front. Immunol., vol. 3, no. JUL, pp. 1-12, 2012.

1. A multispecific antibody comprising a first antigen binding moiety, which specifically binds to an immune checkpoint protein on intratumor T cells and a second antigen binding moiety which specifically binds to a conventional dendritic cell 1 (cDC1).

2. The multispecific antibody of claim 1, wherein said immune checkpoint protein is selected from the group consisting of PD-1, CTLA-4, TIGIT, LAG-3, TIM-3, ICOS, BTLA, 4-1BB, GITR and OX-40.

3. The multispecific antibody of claim 1, wherein said first antigen binding moiety prevents the binding between PD-1 of T cells and PDL-1 expressing cells.

4. The multispecific antibody of claim 1, wherein said second antigen binding moiety binds Clec9a.

5. The multispecific antibody of claim 1, wherein said immune checkpoint protein is PD-1.

6. The multispecific antibody of claim 5, wherein said first antigen binding moiety comprises complementary determining regions as set forth in SEQ ID NOs: 1-3 in a heavy chain with an N to C orientation and complementary determining regions as set forth in SEQ ID NOs: 4-6 in a light chain with an N to C orientation.

7. The multispecific antibody of claim 5, wherein said first antigen binding moiety comprises complementary determining regions as set forth in SEQ ID NOs: 11-13 in a heavy chain with an N to C orientation and complementary determining regions as set forth in SEQ ID NOs: 14-16 in a light chain with an N to C orientation.

8. The multispecific antibody of claim 1, wherein said second antigen binding moiety comprises complementary determining regions as set forth in SEQ ID NOs: 21-23 in a heavy chain with an N to C orientation and complementary determining regions as set forth in SEQ ID NOs: 24-26 in a light chain with an N to C orientation.

9. The multispecific antibody of claim 1, wherein said second antigen binding moiety comprises complementary determining regions as set forth in SEQ ID NOs: 31-33 in a heavy chain with an N to C orientation and complementary determining regions as set forth in SEQ ID NOs: 34-36 in a light chain with an N to C orientation.

10. The multispecific antibody of claim 1, wherein an Fc region of said multispecific antibody comprises a mutation that serves to reduce binding of said antibody to FcγRs.

11. The multispecific antibody of claim 1, comprising knobs-into-holes mutations.

12. The multispecific antibody of claim 11, wherein said mutations are in a CH3 domain of a first antibody of said multispecific antibody comprising Y349C/T366S/L368A/Y407V and in a CH3 domain of a second antibody of said multispecific antibody comprising S354C/T366W.

13. The multispecific antibody of claim 1, wherein said first moiety comprises amino acid sequences as set forth in SEQ ID NOs: 7 and 8; or SEQ ID NOs: 17 and 18.

14. The multispecific antibody of claim 1, wherein said first moiety comprises amino acid sequences as set forth in SEQ ID NOs: 27 and 28; or SEQ ID NOs: 37 and 38.

15. The multispecific antibody of claim 1, being a bispecific antibody.

16. The multispecific antibody of claim 1, for use in treating cancer.

17. A pharmaceutical composition comprising the multispecific antibody of claim 1.

18. A nucleic acid encoding a heavy and/or light chain of the multispecific antibody of claim 1.

19. An expression vector comprising the nucleic acid of claim 18.

20. A cell transformed with the expression vector of claim 19.

21. A method of preparing a multispecific antibody comprising:
(a) culturing the cell of claim 20 under conditions which allow the expression of the multispecific antibody; and
(b) isolating the multispecific antibody from the cell.

22. A method of treating cancer in a subject in need thereof, the method comprising administering to the subject a therapeutically effective amount of the pharmaceutical composition of claim 17, thereby treating cancer in the subject.

23. The method of claim 22, wherein a tumor of said cancer is characterized by having a T cell: dendritic cell ratio above a predetermined level.

24. The multispecific antibody of claim 16, wherein the cancer is selected from the group consisting of: bladder cancer, breast cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer.


SEQUENCE LISTING (41 sequences)

SEQ ID NO: 1 - protein, 6 aa, synthetic construct
  REGION 1..6: note = Complementarity-determining region (CDR)
ITSSYR

SEQ ID NO: 2 - protein, 7 aa, synthetic construct
  REGION 1..7: note = Complementarity-determining region (CDR)
INSAGIS

SEQ ID NO: 3 - protein, 13 aa, synthetic construct
  REGION 1..13: note = Complementarity-determining region (CDR)
ARSDNMGTTPFTY

SEQ ID NO: 4 - protein, 11 aa, synthetic construct
  REGION 1..11: note = Complementarity-determining region (CDR)
KSLLYSDGKTY

SEQ ID NO: 5 - skipped

SEQ ID NO: 6 - protein, 8 aa, synthetic construct
  REGION 1..8: note = Complementarity-determining region (CDR)
QQGLEFPT

SEQ ID NO: 7 - protein, 120 aa, synthetic construct
  REGION 1..120: note = RMP1-14 (Anti-PD-1) VH amino acid sequence
EVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSS

SEQ ID NO: 8 - protein, 111 aa, synthetic construct
  REGION 1..111: note = RMP1-14 (Anti-PD-1) VL amino acid sequence
DIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELE

SEQ ID NO: 9 - protein, 469 aa, synthetic construct
  REGION 1..469: note = RMP1-14 (Anti-PD-1) HC Male N297A amino acid sequence
MGWSCIILFLVATATGVHSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 237 aa, synthetic construct
  REGION 1..237: note = RMP1-14 (Anti-PD-1) LC Mono amino acid sequence
MGWSCIILFLVATATGVHSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
  REGION 1..8: note = Complementarity-determining region (CDR)
GFTFSDYF

SEQ ID NO: 12 - protein, 10 aa, synthetic construct
  REGION 1..10: note = Complementarity-determining region (CDR)
IYTKSYNYAT

SEQ ID NO: 13 - protein, 12 aa, synthetic construct
  REGION 1..12: note = Complementarity-determining region (CDR)
TRDGSGYPSLDF

SEQ ID NO: 14 - protein, 6 aa, synthetic construct
  REGION 1..6: note = Complementarity-determining region (CDR)
QLPKYF

SEQ ID NO: 15 - skipped

SEQ ID NO: 16 - protein, 13 aa, synthetic construct
  REGION 1..13: note = Complementarity-determining region (CDR)
FSGYVDSDSKLYV

SEQ ID NO: 17 - protein, 121 aa, synthetic construct
  REGION 1..121: note = J43 (Anti-PD-1) VH amino acid sequence
EVRLLESGGGLVKPEGSLKLSCVASGFTFSDYFMSWVRQAPGKGLEWVAHIYTKSYNYATYYSGSVKGRFTISRDDSRSMVYLQMNNLRTEDTATYYCTRDGSGYPSLDFWGQGTQVTVSS

SEQ ID NO: 18 - protein, 109 aa, synthetic construct
  REGION 1..109: note = J43 (Anti-PD-1) VL amino acid sequence
YELTQPPSASVNVGETVKITCSGDQLPKYFADWFHQRSDQTILQVIYDDNKRPSGIPERISGSSSGTTATLTIRDVRAEDEGDYYCFSGYVDSDSKLYVFGSGTQLTVL

SEQ ID NO: 19 - protein, 470 aa, synthetic construct
  REGION 1..470: note = J43 (Anti-PD-1) HC Male N297A amino acid sequence
MGWSCIILFLVATATGVHSEVRLLESGGGLVKPEGSLKLSCVASGFTFSDYFMSWVRQAPGKGLEWVAHIYTKSYNYATYYSGSVKGRFTISRDDSRSMVYLQMNNLRTEDTATYYCTRDGSGYPSLDFWGQGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 235 aa, synthetic construct
  REGION 1..235: note = J43 (Anti-PD-1) LC Mono amino acid sequence
MGWSCIILFLVATATGVHSYELTQPPSASVNVGETVKITCSGDQLPKYFADWFHQRSDQTILQVIYDDNKRPSGIPERISGSSSGTTATLTIRDVRAEDEGDYYCFSGYVDSDSKLYVFGSGTQLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 8 aa, synthetic construct
  REGION 1..8: note = Complementarity-determining region (CDR)
GYTFTNYW

SEQ ID NO: 22 - protein, 9 aa, synthetic construct
  REGION 1..9: note = Complementarity-determining region (CDR)
IHPNSDNTK

SEQ ID NO: 23 - protein, 9 aa, synthetic construct
  REGION 1..9: note = Complementarity-determining region (CDR)
ARFANDGAY

SEQ ID NO: 24 - protein, 11 aa, synthetic construct
  REGION 1..11: note = Complementarity-determining region (CDR)
QSLVHSNGNTY

SEQ ID NO: 25 - skipped

SEQ ID NO: 26 - protein, 9 aa, synthetic construct
  REGION 1..9: note = Complementarity-determining region (CDR)
SQNTHVPYT

SEQ ID NO: 27 - protein, 116 aa, synthetic construct
  REGION 1..116: note = Complementarity-determining region (CDR)
QVQLQQPGAELVKPGASVKLSCKASGYTFTNYWIHWMKQRPGQGLEWIGMIHPNSDNTKYNEKFKAKAILTVDKSSSTAYMQLSSLTSEDSAVYYCARFANDGAYWGQGTLVTVSS

SEQ ID NO: 28 - protein, 112 aa, synthetic construct
  REGION 1..112: note = MARX10 (Anti-XCR1) VL amino acid sequence
DVVVTQTPLSLPVSLGDPASISCKSSQSLVHSNGNTYLHWYLQKPGQSPKLLIYKISNRFSGVPDRFSGSGSGTDFTLKISRVEAEDLGVYFCSQNTHVPYTFGGGTKLEIK

SEQ ID NO: 29 - protein, 484 aa, synthetic construct
  REGION 1..484: note = MARX10 (Anti-XCR1) HC Female N297A amino acid sequence
MGWSCIILFLVATATGQVQLQQPGAELVKPGASVKLSCKASGYTFTNYWIHWMKQRPGQGLEWIGMIHPNSDNTKYNEKFKAKAILTVDKSSSTAYMQLSSLTSEDSAVYYCARFANDGAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDRSEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKSAWSHPQFEK

SEQ ID NO: 30 - protein, 236 aa, synthetic construct
  REGION 1..236: note = MARX10 (Anti-XCR1) LC Female amino acid sequence
MGWSCIILFLVATATGVHSDVVVTQTPLSLPVSLGDPASISCKSSQSLVHSNGNTYLHWYLQKPGQSPKLLIYKISNRFSGVPDRFSGSGSGTDFTLKISRVEAEDLGVYFCSQNTHVPYTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 8 aa, synthetic construct
  REGION 1..8: note = Complementarity-determining region (CDR)
GFTFSNAA

SEQ ID NO: 32 - protein, 10 aa, synthetic construct
  REGION 1..10: note = Complementarity-determining region (CDR)
IRTRPSKYAT

SEQ ID NO: 33 - protein, 11 aa, synthetic construct
  REGION 1..11: note = Complementarity-determining region (CDR)
TPRATEDVPFY

SEQ ID NO: 34 - protein, 12 aa, synthetic construct
  REGION 1..12: note = Complementarity-determining region (CDR)
QSVLYDENKKNY

SEQ ID NO: 35 - skipped

SEQ ID NO: 36 - protein, 9 aa, synthetic construct
  REGION 1..9: note = Complementarity-determining region (CDR)
QQYYDFPPT

SEQ ID NO: 37 - protein, 118 aa, synthetic construct
  REGION 1..118: note = 10B4 (Anti-Clec9a) VH amino acid sequence
QIVESGGGLVQPKESLKISCTASGFTFSNAAIYWVRQTPGKGLEWVGRIRTRPSKYATDYADSVRGRFTISRDDSKSMVYLQMDNLRTEDTAMYYCTPRATEDVPFYWGQGVMVTVSS

SEQ ID NO: 38 - protein, 113 aa, synthetic construct
  REGION 1..113: note = 10B4 (Anti-Clec9a) VL amino acid sequence
DIVMTQTPSSQAVSAGEKVTMNCKSSQSVLYDENKKNYLAWYQQKSGQSPKLLIYWASTGESGVPDRFIGSGSGTDFTLTISSVQAEDLAVYYCQQYYDFPPTFGGGTKLELK

SEQ ID NO: 39 - protein, 486 aa, synthetic construct
  REGION 1..486: note = 10B4 (Anti-Clec9a) HC Female N297A amino acid sequence
MGWSCIILFLVATATGQIVESGGGLVQPKESLKISCTASGFTFSNAAIYWVRQTPGKGLEWVGRIRTRPSKYATDYADSVRGRFTISRDDSKSMVYLQMDNLRTEDTAMYYCTPRATEDVPFYWGQGVMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDRSEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKSAWSHPQFEK

SEQ ID NO: 40 - protein, 237 aa, synthetic construct
  REGION 1..237: note = 10B4 (Anti-Clec9a) LC Female amino acid sequence
MGWSCIILFLVATATGVHSDIVMTQTPSSQAVSAGEKVTMNCKSSQSVLYDENKKNYLAWYQQKSGQSPKLLIYWASTGESGVPDRFIGSGSGTDFTLTISSVQAEDLAVYYCQQYYDFPPTFGGGTKLELKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 41 - protein, 329 aa, Homo sapiens
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK